(19) United States
(12) Patent Application Publication
(10) Pub. No.: US 2017/0072206 A1
(43) Pub. Date: Mar. 16, 2017

(54) ELECTRICAL STIMULATION SYSTEMS SUITABLE FOR SHORT-TERM IMPLANTATION AND METHODS OF MAKING AND USING

(71) Applicant: Boston Scientific Neuromodulation Corporation, Valencia, CA (US)

(72) Inventor: Joshua Dale Howard, Sacramento, CA (US)

(21) Appl. No.: 15/261,677

(22) Filed: Sep. 9, 2016

Related U.S. Application Data
(60) Provisional application No. 62/216,885, filed on Sep. 10, 2015.

(51) Int. Cl. A61N 1/372; A61N 1/378; A61N 1/05

(52) U.S. Cl. CPC A61N 1/37223; A61N 1/05; A61N 1/37264; A61N 1/3787

(57) ABSTRACT

An electrical stimulation system includes an electrical stimulation lead; stimulation circuitry to generate stimulation signals; a substrate with the stimulation circuitry disposed on the substrate and the electrical stimulation lead permanently affixed to at least one of the stimulation circuitry or the substrate or an optional housing; a power source electrically coupled to the stimulation circuitry; and an antenna electrically coupled to the stimulation circuitry to receive at least one of a) power for charging the power source or b) signals for programming the stimulation circuitry.

### CROSS-REFERENCE TO RELATED APPLICATIONS

This application claims the benefit under 35 U.S.C. §119(e) of U.S. Provisional Patent Application Ser. No. 62/216,885, filed Sep. 10, 2015, which is incorporated herein by reference.

### FIELD

The present invention is directed to the area of implantable electrical stimulation systems and methods of making and using the systems. The present invention is also directed to implantable electrical stimulation systems that may be suitable for short- term implantation, as well as methods of making and using the electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is an electrical stimulation system that includes an electrical stimulation lead having a lead body having a distal end portion, a proximal end portion, and a longitudinal length; electrodes disposed along the distal end portion of the lead body; and conductors electrically coupled to the electrodes and extending along the lead body. The electrical stimulation system also includes stimulation circuitry to generate stimulation signals for delivery to the patient through the electrodes of the electrical stimulation lead with the conductors of the electrical stimulation lead electrically coupled to the stimulation circuitry; a flexible substrate with the stimulation circuitry disposed on the flexible substrate and the electrical stimulation lead permanently affixed to at least one of the stimulation circuitry or the flexible substrate; a power source electrically coupled to the stimulation circuitry; and an antenna electrically coupled to the stimulation circuitry to receive at least one of a) power for charging the power source or b) signals for programming the stimulation circuitry.

In at least some embodiments, the flexible substrate is wrapped around at least a portion of the proximal end portion of the lead body of the electrical stimulation lead. In at least some embodiments, the flexible substrate forms a cylinder around at least a portion of the stimulation circuitry. In at least some embodiments, the electrical stimulation system further includes traces or contact pads formed on the flexible substrate with the conductors directly attached to the traces or contact pads. In at least some embodiments, the electrical stimulation system further includes a material encasing the flexible substrate, stimulation circuitry, and power source.

In at least some embodiments, the electrical stimulation system further includes a stylet, wherein the electrical stimulation lead includes a stylet lumen. In at least some embodiments, the flexible substrate forms a cylinder around at least a portion of the stimulation circuitry and the electrical stimulation lead so that the stylet is inserted into the stylet lumen of the electrical stimulation lead and through the cylinder formed by the flexible substrate. In at least some embodiments, the lead defines a stylet access port distal of the flexible substrate and power source for insertion of the stylet into the stylet lumen.

A further embodiment is a method of providing electrical stimulation to a patient that includes implanting the electrical stimulation system described above in the patient; and delivering electrical stimulation signals from the stimulation circuitry to the electrodes of the electrical stimulation lead.

Another embodiment is an electrical stimulation system that includes an electrical stimulation lead having a lead body having a distal end portion, a proximal end portion, and a longitudinal length; electrodes disposed along the distal end portion of the lead body; and conductors electrically coupled to the electrodes and extending along the lead body. The electrical stimulation system also includes a housing disposed around at least a part of the proximal end of the lead body of the electrical stimulation lead; stimulation circuitry disposed within the housing to generate stimulation signals for delivery to the patient through the electrodes of the electrical stimulation lead with the conductors of the electrical stimulation lead electrically coupled to the stimulation circuitry; a substrate disposed within the housing with the stimulation circuitry disposed on the substrate and the electrical stimulation lead permanently affixed to at least one of the housing, the stimulation circuitry, or the substrate; a power source disposed within the housing and electrically coupled to the stimulation circuitry; and an antenna disposed within the housing and electrically coupled to the stimulation circuitry to receive at least one of a) power for charging the power source or b) signals for programming the stimulation circuitry.

In at least some embodiments, the electrical stimulation system further includes traces or contact pads formed on the flexible substrate and the conductors are directly attached to the traces or contact pads. In at least some embodiments, the electrical stimulation system further includes a non-conductive material encasing the substrate, stimulation circuitry, and power source.

In at least some embodiments, the electrical stimulation system further includes a stylet and the electrical stimulation lead includes a stylet lumen. In at least some embodiments, the housing defines a stylet access port through which the stylet is inserted into the stylet lumen of the electrical stimulation lead. In at least some embodiments, the lead defines a stylet access port distal of the housing for insertion of the stylet into the stylet lumen.

In at least some embodiments, the housing includes a dilator tip that slopes and becomes smaller in a distal direction. In at least some embodiments, the conductors of the electrical stimulation lead are permanently affixed to at least one of the stimulation circuitry or the substrate.

A further embodiment is a method of providing electrical stimulation to a patient that includes implanting the electrical stimulation system described above in the patient; and delivering electrical stimulation signals from the stimulation circuitry to the electrodes of the electrical stimulation lead.

Yet another embodiment is an electrical stimulation system that includes a lead extension having a distal end portion, a proximal end portion, and a longitudinal length; a connector defining a port at the distal end of the lead extension; connector contacts disposed in the connector; and conductors electrically coupled to the connector contacts and extending along the lead extension. The electrical stimulation system further includes stimulation circuitry to generate stimulation signals for delivery to the patient through electrodes of an electrical stimulation lead coupled to the lead extension with the conductors of the lead extension electrically coupled to the stimulation circuitry; a flexible substrate with the stimulation circuitry disposed on the flexible substrate and the lead extension permanently affixed to at least one of the stimulation circuitry or the flexible substrate; a power source electrically coupled to the stimulation circuitry; and an antenna electrically coupled to the stimulation circuitry to receive at least one of a) power for charging the power source or b) signals for programming the stimulation circuitry.

A further embodiment is a method of providing electrical stimulation to a patient that includes implanting an electrical stimulation lead in the patient. The electrical stimulation lead has electrodes disposed along a distal end portion of the electrical stimulation lead, terminals disposed along a proximal end portion of the electrical stimulation lead, and conductors extending along the electrical stimulation lead and electrically coupling the electrodes to the terminals. The method also includes electrically coupling the electrical stimulation lead to the connector of the lead extension of the electrical stimulation system described above; implanting the electrical stimulation system in the patient; and delivering electrical stimulation signals from the stimulation circuitry to the electrodes of the electrical stimulation lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a lead electrically coupled to a control module;

FIG. 2A is a schematic view of one embodiment of the control module of FIG. 1 configured and arranged to electrically couple to an elongated device;

FIG. 2B is a schematic view of one embodiment of a lead extension configured and arranged to electrically couple the elongated device of FIG. 2A to the control module of FIG. 1;

FIG. 3 is a schematic side view of a portion of one embodiment of an electrical stimulation system, according to the invention;

FIG. 4 is schematic perspective view of a portion of another embodiment of an electrical stimulation system, according to the invention;

FIG. 5 is schematic perspective view of a portion of a third embodiment of an electrical stimulation system, according to the invention; and

FIG. 6 is a schematic overview of one embodiment of components of an electrical stimulation arrangement, according to the invention.

### DETAILED DESCRIPTION

The present invention is directed to the area of implantable electrical stimulation systems and methods of making and using the systems. The present invention is also directed to implantable electrical stimulation systems that may be suitable for short-term implantation, as well as methods of making and using the electrical stimulation systems.

Suitable implantable electrical stimulation systems include, but are not limited to, a least one lead with one or more electrodes disposed along a distal end of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734;7,761,165; 7,783,359; 7,792,590; 7,809,446; 7,949,395; 7,974,706; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and 2013/0105071; and U.S. patent applications Ser. Nos. 12/177,823 and 13/750,725, all of which are incorporated by reference.

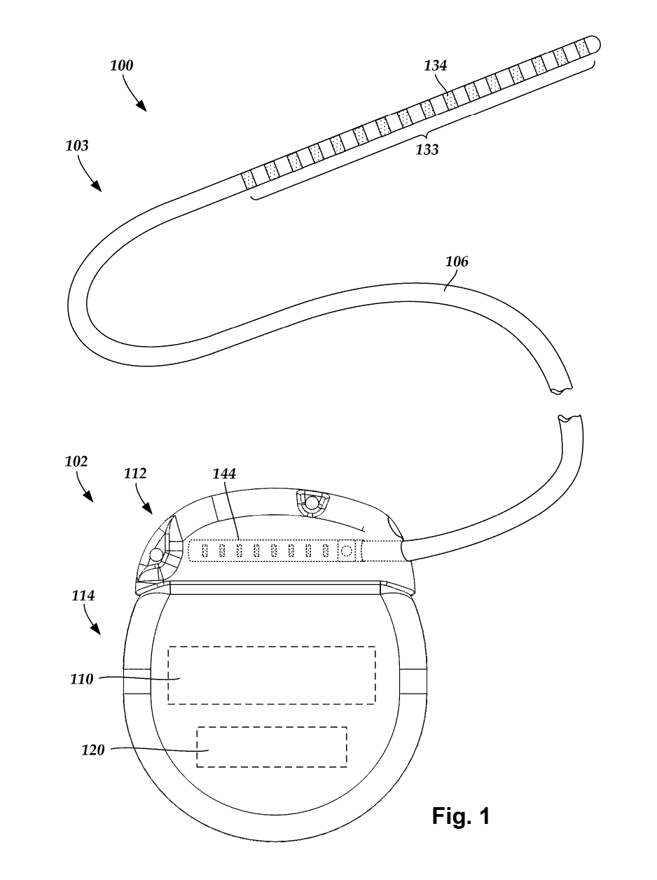

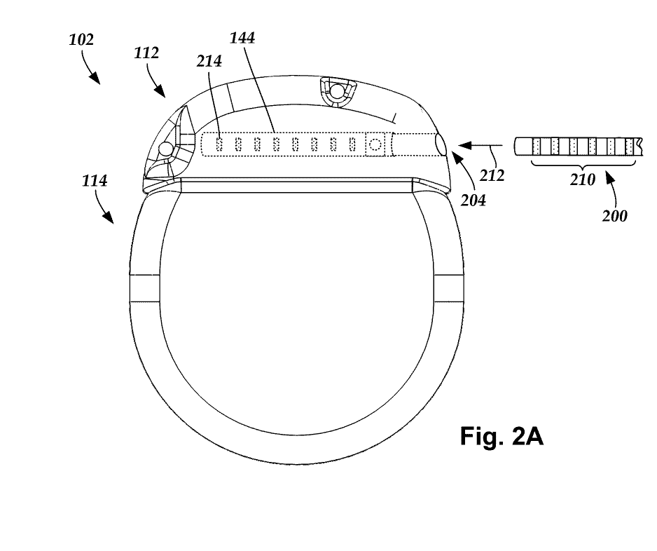

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes one or more lead bodies 106, an array of electrodes 133, such as electrode 134, and an array of terminals (e.g., 210 in FIG. 2A-2B) disposed along the one or more lead bodies 106. In at least some embodiments, the lead is isodiametric along a longitudinal length of the lead body 106.

The lead 103 can be coupled to the control module 102 in any suitable manner. In at least some embodiments, the lead 103 couples directly to the control module 102. In at least some other embodiments, the lead 103 couples to the control module 102 via one or more intermediate devices (200 in FIGS. 2A-2B). For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

The control module 102 typically includes a connector housing 112 and a sealed electronics housing 114. Stimulation circuitry 110 and an optional power source 120 are disposed in the electronics housing 114. A control module connector 144 is disposed in the connector housing 112. The control module connector 144 is configured and arranged to make an electrical connection between the lead 103 and the stimulation circuitry 110 of the control module 102.

The electrical stimulation system or components of the electrical stimulation system, including the lead body 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. The number of electrodes 134 in each array 133 may vary. For example, there can be two, four, six, eight, ten, twelve, fourteen, sixteen, or more electrodes 134. As will be recognized, other numbers of electrodes 134 may also be used.

The electrodes of the lead body 106 are typically disposed in, or separated by, a non-conductive, biocompatible material such as, for example, silicone, polyurethane, polyetheretherketone (“PEEK”), epoxy, and the like or combinations thereof. The lead body 106 may be formed in the desired shape by any process including, for example, molding (including injection molding), casting, and the like. The non-conductive material typically extends from the distal end of the lead body 106 to the proximal end of the lead body 106.

Terminals (e.g., 210 in FIGS. 2A-2B) are typically disposed along the proximal end of the lead body 106 of the electrical stimulation system 100 (as well as any splitters, lead extensions, adaptors, or the like) for electrical connection to corresponding connector contacts (e.g., 214 and 240 in FIG. 2B). The connector contacts are disposed in connectors (e.g., 144 in FIGS. 1-2B; and 222 in FIG. 2B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the lead body 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal end of one or more elongated devices 200 configured and arranged for coupling to one embodiment of the control module connector 144. The one or more elongated devices may include, for example, the lead body 106, one or more intermediate devices (e.g., the lead extension 224 of FIG. 2B, an adaptor, or the like or combinations thereof), or a combination thereof.

The control module connector 144 defines at least one port into which a proximal end of the elongated device 200 can be inserted, as shown by directional arrow 212. In FIG. 2A (and in other figures), the connector housing 112 is shown having one port 204. The connector housing 112 can define any suitable number of ports including, for example, one, two, three, four, five, six, seven, eight, or more ports.

The control module connector 144 also includes a plurality of connector contacts, such as connector contact 214, disposed within each port 204. When the elongated device 200 is inserted into the port 204, the connector contacts 214 can be aligned with a plurality of terminals 210 disposed along the proximal end(s) of the elongated device(s) 200 to electrically couple the control module 102 to the electrodes (134 of FIG. 1) disposed at a distal end of the lead 103. Examples of connectors in control modules are found in, for example, U.S. Pat. Nos. 7,244,150 and 8,224,450, which are incorporated by reference.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 2B, the lead extension connector 222 is shown disposed at a distal end 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

In at least some instances, a large control module, such as the control module 102 illustrated in FIGS. 1-2B, is not desirable. A smaller, more compact control module may be suitable for situations such as, for example, short-term implantation (for example, 1 or 2 weeks, 1, 2, 3, 4, 6, 8, 12, or 18 months), short-term trial (for example, 1 or 2 weeks, 1, 2, 3, 4, 6, 8, 12, or 18 months), clinical studies (for example, for a period of 1 or 2 weeks, 1, 2, 3, 4, 6, 8, 12, or 18 months), or the like. Such a control module may also be useful when a less invasive surgical implantation is desired, recommended, or required. In some instances, a patient or clinician may be willing to charge the control module more frequently if the control module is smaller or the surgery is less invasive. In addition, there may be more options in the body of the patient for implantation of a smaller control module than are available for the larger control module (which is often implanted in the thoracic body cavity or the buttocks due to the size of the device.) A smaller control module may also be less expensive and particularly useful for trials to determine whether electrical stimulation is beneficial. In at least some embodiments, the electrical stimulation system with the smaller control module can be upgraded to an electrical stimulation system such as that illustrated in FIGS. 1-2B if the trial shows sufficient benefit to the patient. In at least some embodiments, the smaller control module may allow for the device to be MRI (magnetic resonance imaging) conditionally safe because of its implant location and size.

In some embodiments, the control module can be made smaller by permanently affixing the lead (or a lead extension) to the control module. For example, the lead can be hardwired to the stimulation circuitry so that the control module does not need a connector and header.

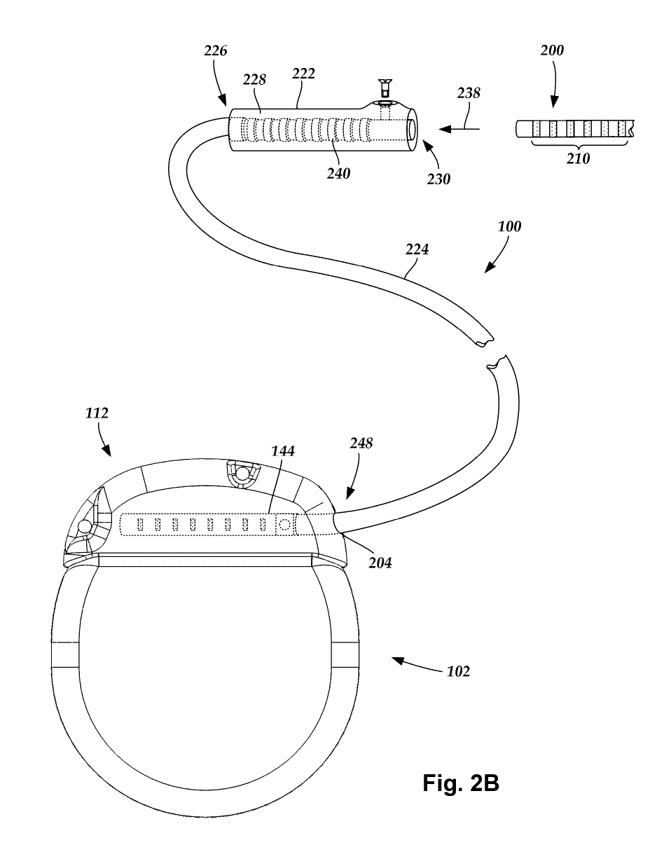

FIG. 3 illustrates one embodiment of an electrical stimulation system with a smaller control module. FIG. 3 illustrates only a part of an attached lead. The electrical stimulation system 300 includes a lead 302, a flexible substrate 304, stimulation circuitry 306 disposed on the flexible substrate, a power source 308, an antenna 310, and an optional stylet 312. In other embodiments, a lead extension can be used in place of lead 302 and then a lead, such as lead 103 of FIG. 1, can be attached to the connector of the lead extension.

The flexible substrate 304 can be any suitable non-conductive substrate such as, for example, substrates used for flex circuits made of non-conductive polymers including, but not limited to, polyimide, polyetheretherketone, polyester, polytetrafluoroethylene, or the like. The stimulation circuitry 306 is attached to the flexible substrate 304 and can include any suitable electronic components for generating the electrical stimulation, as well as wires or traces that, optionally, are formed on the flexible substrate. In at least some embodiments, after attaching the stimulation circuitry 306 to the flexible substrate 304, the flexible substrate can be rolled up into a cylinder with at least some, or even all, of the stimulation circuitry residing within the cylinder.

Any of the leads described above can be used as lead 302. In at least some embodiments, the lead 302 does not include terminals, but rather the conductors that extend from the electrodes of the lead along the lead body can be directly attached or affixed to the flexible substrate 304 (such as a trace or contact pad formed on the flexible substrate) or the stimulation circuitry 306. In at least some embodiments, the lead 302 is permanently affixed to the flexible substrate 304 or stimulation circuitry 306 in contrast to the leads illustrated in FIGS. 1-2B that are removable form the control module. In some embodiments, at least a part of the proximal end portion of the lead 302 can be disposed within a cylinder formed by the flexible substrate 304. In some embodiments, a part of the proximal end portion of the lead 302 may be physically attached to the flexible substrate 304 using adhesive (for example, epoxy) or any other suitable affixation method or components.

Any suitable power source can be used for power source 308 including, but not limited to, a rechargeable battery or chargeable storage capacitors. In at least some embodiments, the power source 308 has a rated lifetime of at least 1, 2, 4, 6, 12, or 18 months. Such a power source 308 would be suitable for relatively short term implantation and may be smaller than power sources used for conventional electrical stimulation systems which, in at least some instances, may have a lifetime of 5 or 10 years or more.

In at least some embodiments, the power source 308 can store sufficient energy that, under average or normal usage conditions, the power source can provide stimulation energy for at least 1, 2, 4, 6, 12, 18, or 24 hours before requiring recharging. In at least some embodiments, such a power source, however, may be limited to providing stimulation energy, under the average or normal usage conditions, for no more than 12, 18, 24, 36, or 48 hours. Such power sources may be smaller than those used in conventional electrical stimulation systems, but require more frequent recharging.

The antenna 310 can be a coil or any other suitable arrangement for receiving energy or signals from an external source, as described in more detail below. In some embodiments, the antenna 310 is electrically coupled to the power source 308 and can be used for recharging the power source. In some embodiments, the antenna 310 is electrically coupled to the stimulation circuitry and can be used to receive signals, such as programming instructions, and provide those signals to the stimulation circuitry 306. In some embodiments, the antenna 310 may also be used for transmission to, for example, transmit data or other information from the stimulation circuitry 306 to an external device, such as programmer or remote control. In at least some embodiments, the antenna 310 can be used to perform two or more of the recharging, receiving, or transmitting functions. The antenna 310 may be configured for receiving or transmitting signals using radiofrequency (RF), near field communications, Bluetooth™, MCIS (Medical Implant Communication Service), or any other suitable frequency, frequency band, or protocol.

In at least some embodiments, the lead 302 has a stylet lumen so that a stylet 312 can be inserted into the proximal end portion of the lead. In at least some embodiments, there is a stylet access port or guide tube within the cylinder formed by the flexible substrate 304 so that the stylet 312 can be inserted into the lead, as illustrated in FIG. 3. In other embodiments, the lead 302 can have an access port distal of the flexible substrate 304 and power source 308 so that the stylet 312 can be inserted into the lead 302 distal to the other components of the electrical stimulation system 300.

In at least some embodiments, the flexible substrate 304, stimulation circuitry 306, and power source 308 can be encased in a material, such as epoxy, parylene, titanium, or any other suitable material that can provide protection for at least an expected implantation lifetime (for example, 6, 12, 18, or 24 months) of the electrical stimulation system 300.

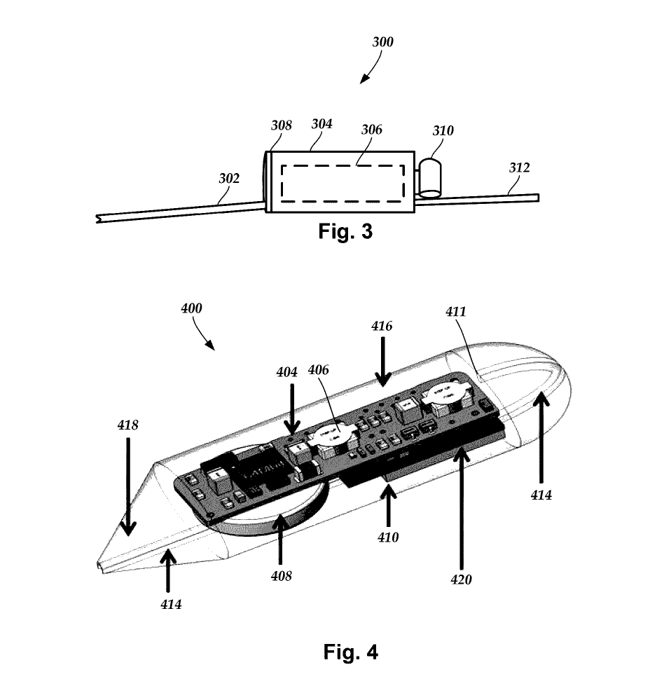

FIG. 4 illustrates another embodiment of an electrical stimulation system 400 that includes a substrate 404, stimulation circuitry 406 disposed on the substrate, a power source 408, an antenna 410, a lead channel 414, and a housing 416. The lead is not illustrated in FIG. 4, but it will be understood that a portion of the lead will reside in the lead channel 414. In other embodiments, a lead extension can be used in place of the lead, with a portion of the lead extension residing in the lead channel 414, and then a lead, such as lead 103 of FIG. 1, can be attached to the connector of the lead extension.

The substrate 404 can be any suitable non-conductive substrate such as, for example, substrates used for electronic circuitry including, but not limited to, polyimide, polyetheretherketone, polyester, polytetrafluoroethylene, or the like. The stimulation circuitry 406 is attached to the substrate 404 and can include any suitable electronic components for generating the electrical stimulation, as well as wires or traces that, optionally, are formed on the flexible substrate.

Any of the leads described above can be used in electrical stimulation system 400. A portion of the lead resides in the lead channel 414 and a remainder of the lead extends out of the housing 416. In at least some embodiments, the lead does not include terminals, but rather the conductors that extend from the electrodes of the lead along the lead body can be directly attached or affixed to the substrate 404 (such as a trace or contact pad formed on the substrate) or the stimulation circuitry 406. In at least some embodiments, the lead is permanently affixed to the housing 416, substrate 404, or stimulation circuitry 406 in contrast to the leads illustrated in FIGS. 1-2B that are removable form the control module. In some embodiments, a part of the proximal end portion of the lead 402 may be physically attached to the housing 416 or substrate 404 using adhesive (for example, epoxy) or any other suitable affixation method or component.

Any suitable power source can be used for power source 408 including, but not limited to, a rechargeable battery or chargeable storage capacitors. In at least some embodiments, the power source 408 has a rated lifetime of at least 1, 2, 4, 6, 12, or 18 months. Such a power source 408 would be suitable for relatively short term implantation and may be smaller than power sources used for conventional electrical stimulation systems which, in at least some instances, may have a lifetime of 5 or 10 years or more.

In at least some embodiments, the power source 408 can store sufficient energy that, under average or normal usage conditions, the power source can provide stimulation energy for at least 1, 2, 4, 6, 12, 18, or 24 hours before requiring recharging. In at least some embodiments, such a power source, however, may be limited to providing stimulation energy, under the average or normal usage conditions, for no more than 12, 18, 24, 36, or 48 hours. Such power sources may be smaller than those used in conventional electrical stimulation systems, but require more frequent recharging.

The antenna 410 can be a coil or any other suitable arrangement for receiving energy or signals from an external source, as described in more detail below. In some embodiments, the antenna 410 is electrically coupled to the power source 408 and can be used for recharging the power source. In some embodiments, the antenna 410 is electrically coupled to the stimulation circuitry and can be used to receive signals, such as programming instructions, and provide those signals to the stimulation circuitry 406. In some embodiments, the antenna 410 may also be used for transmission to, for example, transmit data or other information from the stimulation circuitry 406 to an external device, such as programmer or remote control. In at least some embodiments, the antenna 410 can be used to perform two or more of the recharging, receiving, or transmitting functions. The antenna 410 may be configured for receiving or transmitting signals using radiofrequency (RF), near field communications, Bluetooth™, MCIS (Medical Implant Communication Service), or any other suitable frequency, frequency band, or protocol.

In at least some embodiments, the lead has a stylet lumen so that a stylet can be inserted into the proximal end portion of the lead. In at least some embodiments, there is a stylet access port 411 within the housing 416 so that the stylet can be inserted into the lead. In other embodiments, the lead can have an access port distal of the housing 416 so that the stylet can be inserted into the lead distal to the other components of the electrical stimulation system 400.

In at least some embodiments, the substrate 404 and stimulation circuitry 406, and, optionally, a portion of the lead can be encased in a non-conductive material 420, such as epoxy or parylene, to provide protection or resistance to bodily fluids for at least an expected implantation lifetime (for example, 6, 12, 18, or 24 months) of the electrical stimulation system 400.

The housing 416 can be formed out of any suitable material including, but not limited to, epoxy, titanium, or any other suitable housing material. In at least some embodiments, the housing 416 includes a dilator tip 418 along a distal end of the housing. The dilator tip 418 slopes, and becomes smaller, in the distal direction to assist insertion of the housing 416 and other components into an insertion opening in the patient by dilating the patient tissue.

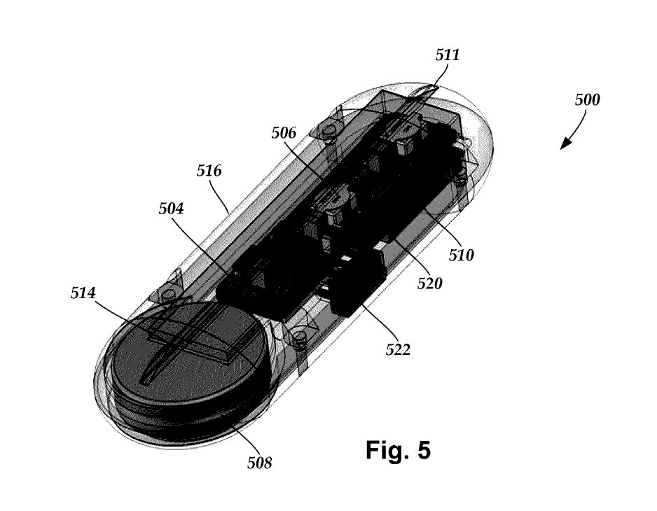

FIG. 5 illustrates another embodiment of an electrical stimulation system 500 that includes a substrate 504, stimulation circuitry 506 disposed on the substrate, a power source 508, an antenna 510, a lead channel 514, and a housing 516. The lead is not illustrated in FIG. 5, but it will be understood that a portion of the lead will reside in the lead channel 514. In other embodiments, a lead extension can be used in place of the lead, with a portion of the lead extension residing in the lead channel 514, and then a lead, such as lead 103 of FIG. 1, can be attached to the connector of the lead extension.

The substrate 504 can be any suitable non-conductive substrate such as, for example, substrates used for electronic circuitry including, but not limited to, polyimide, polyetheretherketone, polyester, polytetrafluoroethylene, or the like. The stimulation circuitry 506 is attached to the substrate 504 and can include any suitable electronic components for generating the electrical stimulation, as well as wires or traces that, optionally, are formed on the flexible substrate.

Any of the leads described above can be used in electrical stimulation system 500. A portion of the lead resides in the lead channel 514 and a remainder of the lead extends out of the housing 516. In at least some embodiments, the lead does not include terminals, but rather the conductors that extend from the electrodes of the lead along the lead body can be directly attached or affixed to the substrate 504 (such as a trace or contact pad formed on the substrate) or the stimulation circuitry 506. In at least some embodiments, the lead is permanently affixed to the housing 516, substrate 504, or stimulation circuitry 506 in contrast to the leads illustrated in FIGS. 1-2B that are removable form the control module. In some embodiments, a part of the proximal end portion of the lead 502 may be physically attached to the housing 516 or substrate 504 using adhesive (for example, epoxy) or any other suitable affixation method or component.

Any suitable power source can be used for power source 508 including, but not limited to, a rechargeable battery or chargeable storage capacitors. In at least some embodiments, the power source 508 has a rated lifetime of at least 1, 2, 4, 6, 12, or 18 months. Such a power source 508 would be suitable for relatively short term implantation and may be smaller than power sources used for conventional electrical stimulation systems which, in at least some instances, may have a lifetime of 5 or 10 years or more.

In at least some embodiments, the power source 508 can store sufficient energy that, under average or normal usage conditions, the power source can provide stimulation energy for at least 1, 2, 4, 6, 12, 18, or 24 hours before requiring recharging. In at least some embodiments, such a power source, however, may be limited to providing stimulation energy, under the average or normal usage conditions, for no more than 12, 18, 24, 36, or 48 hours. Such power sources may be smaller than those used in conventional electrical stimulation systems, but require more frequent recharging.

The antenna 510 can be a coil or any other suitable arrangement for receiving energy or signals from an external source, as described in more detail below. In some embodiments, the antenna 510 is electrically coupled to the power source 508 and can be used for recharging the power source. In some embodiments, the antenna 510 is electrically coupled to the stimulation circuitry and can be used to receive signals, such as programming instructions, and provide those signals to the stimulation circuitry 506. In some embodiments, the antenna 510 may also be used for transmission to, for example, transmit data or other information from the stimulation circuitry 506 to an external device, such as programmer or remote control. In at least some embodiments, the antenna 510 can be used to perform two or more of the recharging, receiving, or transmitting functions. The antenna 510 may be configured for receiving or transmitting signals using radiofrequency (RF), near field communications, Bluetooth™, MCIS (Medical Implant Communication Service), or any other suitable frequency, frequency band, or protocol.

In at least some embodiments, the lead has a stylet lumen so that a stylet can be inserted into the proximal end portion of the lead. In at least some embodiments, there is a stylet access port 511 within the housing 516 so that the stylet can be inserted into the lead. In other embodiments, the lead can have an access port distal of the housing 516 so that the stylet can be inserted into the lead distal to the other components of the electrical stimulation system 500.

In at least some embodiments, the substrate 504 and stimulation circuitry 506, and, optionally, a portion of the lead can be encased in a non-conductive material 520, such as epoxy or parylene, to provide protection or resistance to bodily fluids for at least an expected implantation lifetime (for example, 6, 12, 18, or 24 months) of the electrical stimulation system 500.

The housing 516 can be formed out of any suitable material including, but not limited to, epoxy, titanium, or any other suitable housing material. In the illustrated embodiment, the housing 516 has a clam-shell arrangement and may, at least in some embodiments, be made in two parts so that the other components of the electrical stimulation system 500 can be inserted into one or both parts and then the two parts coupled together using adhesive, welding, or any other affixation method or components.

The electrical stimulation system 500 also includes an optional switch 522 that can be used to turn on and off the stimulation circuitry 506. This may be useful so that the electrical stimulation system 500 is only turned on when the electrical stimulation system is implanted or about to be implanted.

The electrical stimulation systems 300, 400, and 500 can be implanted into a patient. One method of implantation includes using a lateral release lead introducer such as those described in U.S. Patent Applications Publication Nos. 2011/0224680; 2014/0039586; 2014/0276927; 2015/0073431; and 2015/0073432 and U.S. Provisional Patent Application Ser. No. 62/153,844, all of which are incorporated herein by reference in their entireties. A lateral release lead introducer can include a multi-piece insertion needle that enables the lead of the electrical stimulation system to be laterally separated by splitting apart the needle. Thus, the lead can be implanted using the needle of the introducer while the remainder of the components of the electrical stimulation system are attached to the lead.

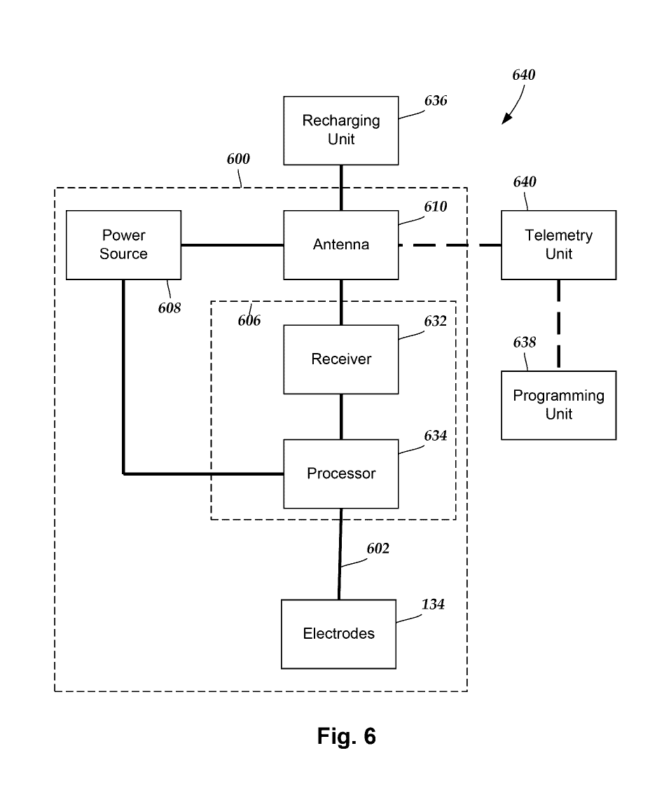

FIG. 6 is a schematic overview of one embodiment of components of an electrical stimulation arrangement 640 that includes an electrical stimulation system 600 with a lead 602, stimulation circuitry 606, a power source 608, and an antenna 610. The electrical stimulation system can be, for example, any of the electrical stimulation systems 300, 400, or 500 described above. It will be understood that the electrical stimulation arrangement can include more, fewer, or different components and can have a variety of different configurations including those configurations disclosed in the stimulator references cited herein.

If the power source 608 is a rechargeable battery or chargeable capacitor, the power source may be recharged/charged using the antenna 610, if desired. Power can be provided for recharging/charging by inductively coupling the power source 608 through the antenna 610 to a recharging unit 636 external to the user. Examples of such arrangements can be found in the references identified above.

In one embodiment, electrical current is emitted by the electrodes (such as electrodes 134 in FIG. 1) on the lead 602 to stimulate nerve fibers, muscle fibers, or other body tissues near the electrical stimulation system. The stimulation circuitry 606 can include, among other components, a processor 634 and a receiver 632. The processor 634 is generally included to control the timing and electrical characteristics of the electrical stimulation system. For example, the processor 634 can, if desired, control one or more of the timing, frequency, strength, duration, and waveform of the pulses. In addition, the processor 634 can select which electrodes can be used to provide stimulation, if desired. In some embodiments, the processor 634 selects which electrode(s) are cathodes and which electrode(s) are anodes. In some embodiments, the processor 634 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 638 that, for example, allows modification of pulse characteristics. In the illustrated embodiment, the processor 634 is coupled to a receiver 632 which, in turn, is coupled to the antenna 610. This allows the processor 634 to receive instructions from an external source to, for example, direct the pulse characteristics and the selection of electrodes, if desired.

In one embodiment, the antenna 610 is capable of receiving signals (e.g., RF signals) from an external telemetry unit 640 that is programmed by the programming unit 638. The programming unit 638 can be external to, or part of, the telemetry unit 640. The telemetry unit 640 can be a device that is worn on the skin of the user or can be carried by the user and can have a form similar to a pager, cellular phone, or remote control, if desired. As another alternative, the telemetry unit 640 may not be worn or carried by the user but may only be available at a home station or at a clinician's office. The programming unit 638 can be any unit that can provide information to the telemetry unit 640 for transmission to the electrical stimulation system 600. The programming unit 638 can be part of the telemetry unit 640 or can provide signals or information to the telemetry unit 640 via a wireless or wired connection. One example of a suitable programming unit is a computer operated by the user or clinician to send signals to the telemetry unit 640.

The signals sent to the processor 634 via the antenna 610 and the receiver 632 can be used to modify or otherwise direct the operation of the electrical stimulation system 600. For example, the signals may be used to modify the pulses of the electrical stimulation system such as modifying one or more of pulse duration, pulse frequency, pulse waveform, and pulse strength. The signals may also direct the electrical stimulation system 600 to cease operation, to start operation, to start charging the battery, or to stop charging the battery.

Optionally, the electrical stimulation system 600 may include a transmitter (not shown) coupled to the processor 634 and the antenna 610 for transmitting signals back to the telemetry unit 640 or another unit capable of receiving the signals. For example, the electrical stimulation system 600 may transmit signals indicating whether the electrical stimulation system 600 is operating properly or not or indicating when the battery needs to be charged or the level of charge remaining in the battery. The processor 634 may also be capable of transmitting information about the pulse characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation system, comprising:
an electrical stimulation lead comprising a lead body having a distal end portion, a proximal end portion, and a longitudinal length; a plurality of electrodes disposed along the distal end portion of the lead body; and a plurality of conductors electrically coupled to the electrodes and extending along the lead body;
stimulation circuitry configured and arranged to generate stimulation signals for delivery to the patient through the electrodes of the electrical stimulation lead, wherein the conductors of the electrical stimulation lead are electrically coupled to the stimulation circuitry;
a flexible substrate, wherein the stimulation circuitry is disposed on the flexible substrate, wherein the electrical stimulation lead is permanently affixed to at least one of the stimulation circuitry or the flexible substrate;
a power source electrically coupled to the stimulation circuitry; and
an antenna electrically coupled to the stimulation circuitry and configured and arranged to receive at least one of a) power for charging the power source or b) signals for programming the stimulation circuitry.

2. The electrical stimulation system of claim 1, wherein the flexible substrate is wrapped around at least a portion of the proximal end portion of the lead body of the electrical stimulation lead.

3. The electrical stimulation system of claim 1, wherein the flexible substrate forms a cylinder around at least a portion of the stimulation circuitry.

4. The electrical stimulation system of claim 1, further comprising traces or contact pads formed on the flexible substrate, wherein the conductors are directly attached to the traces or contact pads.

5. The electrical stimulation system of claim 1, further comprising a stylet, wherein the electrical stimulation lead comprises a stylet lumen.

6. The electrical stimulation system of claim 5, wherein the flexible substrate forms a cylinder around at least a portion of the stimulation circuitry and the electrical stimulation lead, wherein the stylet is inserted into the stylet lumen of the electrical stimulation lead and through the cylinder formed by the flexible substrate.

7. The electrical stimulation system of claim 5, wherein the lead defines a stylet access port distal of the flexible substrate and power source and configured and arranged for insertion of the stylet into the stylet lumen.

8. The electrical stimulation system of claim 1, further comprising a material encasing the flexible substrate, stimulation circuitry, and power source.

9. An electrical stimulation system, comprising:
an electrical stimulation lead comprising a lead body having a distal end portion, a proximal end portion, and a longitudinal length; a plurality of electrodes disposed along the distal end portion of the lead body; and a plurality of conductors electrically coupled to the electrodes and extending along the lead body;
a housing disposed around at least a part of the proximal end of the lead body of the electrical stimulation lead;
stimulation circuitry disposed within the housing and configured and arranged to generate stimulation signals for delivery to the patient through the electrodes of the electrical stimulation lead, wherein the conductors of the electrical stimulation lead are electrically coupled to the stimulation circuitry;
a substrate disposed within the housing, wherein the stimulation circuitry is disposed on the substrate, wherein the electrical stimulation lead is permanently affixed to at least one of the housing, the stimulation circuitry, or the substrate;
a power source disposed within the housing and electrically coupled to the stimulation circuitry; and
an antenna disposed within the housing and electrically coupled to the stimulation circuitry and configured and arranged to receive at least one of a) power for charging the power source or b) signals for programming the stimulation circuitry.

10. The electrical stimulation system of claim 9, further comprising traces or contact pads formed on the flexible substrate, wherein the conductors are directly attached to the traces or contact pads.

11. The electrical stimulation system of claim 9, further comprising a stylet, wherein the electrical stimulation lead comprises a stylet lumen.

12. The electrical stimulation system of claim 11, wherein the housing defines a stylet access port through which the stylet is inserted into the stylet lumen of the electrical stimulation lead.

13. The electrical stimulation system of claim 11, wherein the lead defines a stylet access port distal of the housing and configured and arranged for insertion of the stylet into the stylet lumen.

14. The electrical stimulation system of claim 9, further comprising a non-conductive material encasing the substrate, stimulation circuitry, and power source.

15. The electrical stimulation system of claim 9, wherein the housing comprises a dilator tip that slopes and becomes smaller in a distal direction.

16. The electrical stimulation system of claim 9, wherein the conductors of the electrical stimulation lead are permanently affixed to at least one of the stimulation circuitry or the substrate.

17. An electrical stimulation system, comprising:
a lead extension having a distal end portion, a proximal end portion, and a longitudinal length; a connector defining a port at the distal end of the lead extension; a plurality of connector contacts disposed in the connector; and a plurality of conductors electrically coupled to the connector contacts and extending along the lead extension;
stimulation circuitry configured and arranged to generate stimulation signals for delivery to the patient through electrodes of an electrical stimulation lead coupled to the lead extension, wherein the conductors of the lead extension are electrically coupled to the stimulation circuitry;
a flexible substrate, wherein the stimulation circuitry is disposed on the flexible substrate, wherein the lead extension is permanently affixed to at least one of the stimulation circuitry or the flexible substrate;
a power source electrically coupled to the stimulation circuitry; and
an antenna electrically coupled to the stimulation circuitry and configured and arranged to receive at least one of a) power for charging the power source or b) signals for programming the stimulation circuitry.

18. A method of providing electrical stimulation to a patient, the method comprising:
implanting the electrical stimulation system of claim 1 in the patient; and
delivering electrical stimulation signals from the stimulation circuitry to the electrodes of the electrical stimulation lead.

19. A method of providing electrical stimulation to a patient, the method comprising:
implanting the electrical stimulation system of claim 9 in the patient; and
delivering electrical stimulation signals from the stimulation circuitry to the electrodes of the electrical stimulation lead.

20. A method of providing electrical stimulation to a patient, the method comprising:
implanting an electrical stimulation lead in the patient, the electrical stimulation lead comprising electrodes disposed along a distal end portion of the electrical stimulation lead, terminals disposed along a proximal end portion of the electrical stimulation lead, and conductors extending along the electrical stimulation lead and electrically coupling the electrodes to the terminals;
electrically coupling the electrical stimulation lead to the connector of the lead extension of the electrical stimulation system of claim 17;
implanting the electrical stimulation system in the patient; and
delivering electrical stimulation signals from the stimulation circuitry to the electrodes of the electrical stimulation lead.
